(19) United States
(12) Patent Application Publication
(10) Pub. No.: US 2008/0058653 A1
(43) Pub. Date: Mar. 6, 2008

(54) Blood pressure cuff mate

(76) Inventor: Darlene Douglas, North Las Vegas, NV (US)

Correspondence Address: DARLENE DOUGLAS, 3613 CASA CORONA AVE., NORTH LAS VEGAS, NV, 89031, US

(21) Appl. No.: 11/504,521

(22) Filed: Aug. 15, 2006

(51) Int. Cl. A61B 5/02

(52) U.S. Cl. 600/490

(57) ABSTRACT

The purpose of the blood pressure cuff mate is to aid in preventing the spread of infection or cross contamination by multiple person to person contact by using the same blood pressure cuff.

The blood pressure cuff mate is primarily used in medical settings, i.e., hospitals, clinics, long term care facilities, and health fairs.

The blood pressure cuff mate is a non hypo_non permeanable fabric material which fits securely over the fore arm or leg, during the taking of ones blood pressure.

The blood pressure cuff mate is a barrier between the skin and the actual blood pressure cuff.

1. I claim that the blood pressure cuff mate, when used is an effective tool in aiding in preventing the spread of bacteria and or infections associated with person to person contact.
